(19) United States
(12) Patent Application Publication
(10) Pub. No.: US 2025/0099954 A1
(43) Pub. Date: Mar. 27, 2025

(54) IMPROVED FLOW CELLS, SYSTEMS, AND METHODS

(71) Applicant: MGI Tech Co., Ltd., Shenzhen (CN)

(72) Inventors: Jon Bartman, Mountain View, CA (US); Fang-Wei Liu, San Jose, CA (US); Victoria Ng, San Jose, CA (US); Xianyong Gu, Shenzhen (CN); Bing Du, Shenzhen (CN)

(21) Appl. No.: 18/570,781

(22) PCT Filed: May 16, 2023
(86) PCT No.: PCT/CN2023/094534

§ 371 Date: Dec. 15, 2023

(51) Int. Cl. B01L 3/00; G01N 21/05

(52) U.S. Cl. CPC B01L 3/502715; B01L 3/502784; G01N 21/05; B01L 2300/0877

(57) ABSTRACT

A flow cell including a substrate that is configured to support an analyte array, a cover, and an adhesive arrangement spacing the cover from the substrate to define a fluid gap between the substrate and cover. The flow cell also includes an inlet in fluid communication with the fluid gap and an outer perimeter. In one example implementation, fluid enters the flow cell at the inlet, flows through the fluid gap, and exits the flow cell at the outer perimeter between the substrate and the cover; with adhesive gaps in the adhesive arrangement at the outer perimeter of the flow cell allowing fluid to exit from the fluid gap.

### BACKGROUND

Many current nucleic acid sequencing systems and processes are resource intensive, requiring significant amounts of reagents and a significant time to complete a sequencing process. Massively parallel systems and processes have been developed in an attempt to more efficiently use resources; however, there remains room for improvement.

### SUMMARY

This patent discloses several examples of improved flow cells, systems, and methods.

In one example, a flow cell system includes: (a) a flow cell, the flow cell including: (i) a substrate, the substrate configured to support an analyte array; (ii) a cover spaced from the substrate, the substrate and the cover defining a fluid gap between the substrate and cover; (iii) an inlet in fluid communication with the fluid gap; and (iv) an outer perimeter of the flow cell; (b) the flow cell system being configured such that fluid enters the flow cell at the inlet, flows through the fluid gap, and exits the flow cell at the outer perimeter between the substrate and the cover.

In this example, the flow cell system may be configured such that fluid exits the flow cell at the outer perimeter of the flow cell between the substrate and the cover such that fluid drops down from the outer perimeter as droplets.

In this example, the outer perimeter of the flow cell may be at least partially open.

In this example, the outer perimeter may be at least 2.5% open.

In this example, the outer perimeter may be at least 5% open.

In this example, the outer perimeter of the flow cell may be open.

In this example, the outer perimeter of the flow cell may be frameless.

In this example, the flow cell may further include a spacer spacing the substrate and the cover apart to define the fluid gap.

In this example, the spacer may be a plurality of spacers, at least some of the spacers being generally aligned towards a central area of the flow cell.

In this example, at least some of the spacers may extend radially from the central area of the flow cell.

In this example, the spacer may be an adhesive arrangement spacing the substrate and the cover apart to define the fluid gap and adhering the substrate to the cover.

In this example, the inlet may be located in the central area of the flow cell.

In this example, the system may also include a common collector, the common collector positioned below the outer perimeter of the flow cell and configured to collect droplets that drop down from the outer perimeter of the flow cell.

In this example, the common collector may include a plurality of common collection cups configured to collect droplets that drop down from the outer perimeter of the flow cell.

In this example, the common collection cups may be vertically spaced from the flow cell by a drop gap.

In this example, the common collection cups may each include a sloped side wall, wherein a portion of the sloped side wall below the outer perimeter of the flow cell is vertically spaced from the flow cell by a drop gap.

In this example, at least some of the common collection cups may each comprise a sloped wall configured to direct the collected droplets to a fluid conduit.

In this example, the collection channel may be a plurality of fluid conduits.

In this example, the sloped wall may have a slope that is greater than 30 degrees.

In another example, a flow cell system includes (a) a flow cell, the flow cell having: (i) a substrate, the substrate configured to support an analyte array; (ii) a cover; (iii) an adhesive arrangement spacing the cover from the substrate to define a fluid gap between the substrate and cover; (iv) an inlet in fluid communication with the fluid gap; and (v) an outer perimeter of the flow cell; (b) wherein the flow cell system is configured such that fluid enters the flow cell at the inlet, flows through the fluid gap, and exits the flow cell at the outer perimeter between the substrate and the cover; wherein adhesive gaps in the adhesive arrangement at the outer perimeter of the flow cell allow fluid to exit from the fluid gap.

In this example, there may be at least five separate adhesive gaps in the adhesive arrangement at the outer perimeter of the flow cell that allow fluid to exit from the fluid gap.

In this example, there may be at least ten separate adhesive gaps in the adhesive arrangement at the outer perimeter of the flow cell allow fluid to exit from the fluid gap.

In another example, a flow cell system includes: (a) a flow cell, the flow cell having: (i) a substrate, the substrate having a surface configured to support an analyte array, the surface configured to support the analyte array having a surface area of at least 150 cm²; (ii) a cover spaced from the substrate, the substrate and the cover defining a fluid gap between the substrate and cover, the fluid gap having a fluid gap volume that is less than 1.5 mL; (iii) an inlet in fluid communication with the fluid gap; and (iv) an outer perimeter of the flow cell; (b) the flow cell system is configured such that fluid enters the flow cell at the inlet, flows through the fluid gap, and exits the flow cell at the outer perimeter between the substrate and the cover.

In this example, the surface configured to support the analyte array has a surface area of at least 225 cm² and the fluid gap has a fluid gap volume that is less than 1 mL.

In this example, the surface configured to support the analyte array may have a a surface area of at least 300 cm² and the fluid gap may have a fluid gap volume that is less than 0.75 ml.

In another example, a flow cell system may include: (a) a flow cell, the flow cell having: (i) a substrate, the substrate configured to support an analyte array; (ii) a cover spaced from the substrate, the substrate and the cover defining a fluid gap between the substrate and cover; (iii) an inlet in fluid communication with the fluid gap; and (iv) an outer perimeter of the flow cell; (b) the flow cell system is configured such that fluid enters the flow cell at the inlet, flows through the fluid gap, and exits the flow cell at the outer perimeter of the flow cell such that the fluid drops down from the outer perimeter as droplets.

In this example, the outer perimeter of the flow cell may be open.

In this example, the outer perimeter of the flow cell may be frameless.

In this example, the flow cell may also include a spacer spacing the substrate and the cover apart to define the fluid gap.

In this example, the spacer may be a plurality of spacers, the spacers may be generally aligned towards a central area of the flow cell.

In this example, the spacers may extend radially from the central area of the flow cell.

In this example, the inlet may be located in the central area of the flow cell.

In this example, the outer portions of the spacers are inset relative to the outer perimeter of the flow cell.

In this example, the system may also include a collection funnel, the collection funnel positioned below the outer perimeter of the flow cell and configured to collect droplets that drop down from the outer perimeter of the flow cell.

In this example, the collection funnel may be a plurality of collection funnels configured to collect droplets that drop down from the outer perimeter of the flow cell.

In this example, the collection funnels may be vertically spaced from the flow cell by a drop gap.

In this example, at least some of the collection funnels may each have a sloped wall configured to direct the collected droplets to a collection channel.

In this example, the sloped wall may have a slope that is greater than 30 degrees.

In this example, the collection channel may slope away from the collection funnel.

In this example, the collection channel may slope away from the collection funnel at a collection channel slope that is greater than 5 degrees.

In this example, the flow cell system may be actuatable between a plurality of states including a first state in which the collection funnels are configured to direct collected droplets along a waste pathway and a second state in which the collection funnels are configured to direct collected droplets along a recycling pathway.

In this example, the system may also include a funnel member and a fluidic channel member, the collection funnels may be formed in the funnel member, a plurality of fluidic channels may be formed in the fluidic channel member, and actuating the flow cell system between the plurality of states may involve repositioning the funnel member relative to the fluidic channel member.

In this example, the flow cell system may be actuatable between a plurality of states including a first state in which the collection funnels are configured to direct collected droplets along a waste pathway, a second state in which the collection funnels are configured to direct collected droplets along a first reagent type recycling pathway, and a third state in which the collection funnels are configured to direct collected droplets along a second reagent type recycling pathway.

In this example, the system may also include a sorting sub-system configured to sort droplets collected from the outer perimeter of the flow cell into at least a first group and a second group.

In this example, the sorting sub-system may direct droplets sorted into the first group from a common droplet path to a recycling droplet path of the flow cell system, wherein the sorting sub-system directs droplets sorted into the second group from the common droplet path to a waste droplet path of the flow cell system.

In this example, the system may also include a sorting sub-system configured to sort droplets collected from the outer perimeter of the flow cell into at least a first group, a second group, and a third group.

In this example, the sorting sub-system may direct droplets sorted into the first group from a common droplet path to a first reagent type recycling droplet path of the flow cell system, wherein the sorting sub-system directs droplets sorted into the second group from the common droplet path to a second reagent type recycling droplet path of the flow cell system, wherein the sorting sub-system directs droplets sorted into the third group from the common droplet path to a waste droplet path of the flow cell system.

In this example, the system may also include a vacuum sub-system configured to generate a reduced pressure at the outer perimeter of the flow cell in order to reduce resistance to fluid flow through the fluid gap.

In this example, the system may also include a vacuum chamber, the flow cell positioned inside the vacuum chamber.

In this example, the cover of the flow cell may have an inner surface and an outer surface, the inner surface facing the fluid gap; and wherein the vacuum sub-system may be further configured to generate a reduced pressure at the outer surface of the cover.

In this example, the vacuum sub-system may be configured to generate the reduced pressure at the outer surface of the cover to resist collapse of the cover during filling of the fluid gap of the flow cell.

In this example, during operation of the vacuum sub-system, the generated pressure at the outer surface of the cover may be less than the generated pressure at the outer perimeter of the flow cell; and during operation of the vacuum sub-system, the generated pressure at the outer perimeter of the flow cell may be less than the generated pressure at the inlet of the flow cell.

In this example, the vacuum sub-system may include one or more vacuum pumps configured to generate the reduced pressure at the outer perimeter of the flow cell and configured to generate the reduced pressure at the outer surface of the cover.

In this example, the system may also include an actuator configured to spin the flow cell about a vertical axis to facilitate flow of a fluid from the inlet to the outer perimeter.

In this example, the flow cell inlet may be a centrally located opening extending through the flow cell cover.

In this example, the flow cell may also include a plurality of spacers spacing the substrate and the cover apart to define the fluid gap, the spacers extending radially from a central area of the flow cell.

In this example, the flow cell may also include one or more spacers spacing the substrate and the cover apart to define the fluid gap, the one or more spacers extending in a spiral from a central area of the flow cell.

In another example, a flow cell system includes: (a) a flow cell, the flow cell having: (i) a substrate, the substrate configured to support an analyte array; (ii) a cover spaced from the substrate, the substrate and the cover defining a fluid gap between the substrate and cover; (iii) an inlet in fluid communication with the fluid gap; and (iv) an outlet in fluid communication with the fluid gap; and (b) a vacuum sub-system configured to generate a reduced pressure at the outlet in fluid communication with the fluid gap in order to reduce resistance to fluid flow through the fluid gap.

In this example, the system may further include a vacuum chamber, the flow cell positioned inside the vacuum chamber.

In this example, the cover may have an inner surface and an outer surface, the inner surface facing the fluid gap; and wherein the vacuum sub-system may be further configured to generate a reduced pressure at the outer surface of the cover.

In this example, during operation of the vacuum sub-system, the pressure at the outer surface of the cover may be less than the pressure at the outlet in fluid communication with the fluid gap; and wherein during operation of the vacuum sub-system, the pressure at the outlet in fluid communication with the fluid gap may be less than the pressure at the inlet in fluid communication with the fluid gap.

In this example, the outlet in fluid communication with the fluid gap may be one or more discrete fluid channels extending through the substrate of the flow cell.

In this example, the outlet in fluid communication with the fluid gap may be an open outer perimeter of the flow cell.

### BRIEF DESCRIPTION OF FIGURES

FIG. 1 shows an example of a flow cell with a flow gap.

FIG. 2 shows an example process flow for the flow cell of FIG. 1.

FIGS. 3-6 illustrate an example of a frameless flow cell system.

FIGS. 7-10 illustrate examples of recycling sub-systems for flow cell systems.

FIGS. 11-13 illustrate examples of a frameless flow cell useable in a vacuum chamber.

FIGS. 14-16 illustrate examples of spin coating flow cells.

FIGS. 17-25 illustrate another example of a flow cell system, including a frameless flow cell and a fluid recycling system.

FIGS. 26-28 show a simulation of filling and washing cycles for a flow cell.

### DETAILED DESCRIPTION OF FIGURES

### Flow Cell

The flow cells described in the following examples may be used in a wide variety of implementations, including systems where larger surface area flow cells are desired while at the same time minimizing reagent consumption.

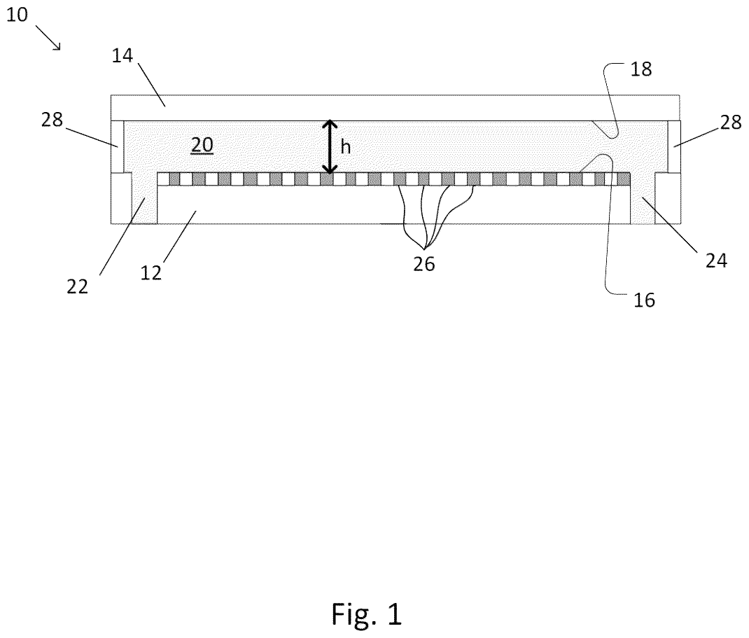

In some implementations, the flow cells may be used as part of a sequencing system for analyzing nucleic acid material such as DNA or RNA, or other biological or non-biological/synthetic material to be analyzed. The flow cell substrate may include an array of analyte attachment sites for attaching nucleic acid fragments or other analyte to the flow cell substrate (e.g. discrete attachment sites 26 schematically shown in FIG. 1—which may be positively charged features on the substrate separated and spaced apart by negatively charged or neutral areas of the substrate). In some implementations, the number of discrete attachment sites may be arranged in arrays that include up to millions or billions of discrete sites, spaced at pitches that may be on the order of tens or hundreds of nanometers.

The nucleic acid fragments attached to the flow cell substrate may be imaged by an optical imaging system or otherwise analyzed. For example, DNA templates may be immobilized at greater than 10e7 attachment sites in an array on the flow cell substrate. In this example, a nucleic acid sequencing method may involve carrying out greater than 400 sequencing cycles. In each cycle, single nucleotides (e.g., adenine, guanine, thymine, and cytosine) may be flowed across the substrate through the fluid gap 20 and incorporated (into a growing strand) at each site where there is a complementary nucleotide base. In one approach, each of the four different nucleotides may be labeled with a different color fluorescent dye or bound by a dye-labeled antibody. In each sequencing cycle, a light source (e.g., a laser) may illuminate the spots (e.g., in series), causing the dye to emit light corresponding to the respective colors. The color emitted at each spot from one of the four dyes may be detected by a camera (e.g., a time delay integration charge-coupled device (TDI-CCD) camera or a similar camera), and the imaging system may thereby record, for each spot, the detection of a nucleotide corresponding to the detected color. Persons knowledgeable in the art will be aware of variations in sequencing methods including variations in template type (see, e.g., Huang et al., 2017, Gigascience 6:1-9; Mardis et al., 2013, Annu Rev Anal Chem 6:287-303), labeling systems (see, e.g., WO2018129214) and labeling strategies (see, e.g., U.S. Pat. No. 9,523,125).

The attachment sites 26 on the substrate 12 of flow cell 10 may be fabricated by well-known lithography tools, such as 248-nm KrF (krypton fluoride), 193-nm ArF (argon-fluoride) lithography systems, or e-beam lithography systems. The arrays are typically separated with spaces between each other in ultra-high density, high density, medium density, or low density. At ultra-high density, separation is less than 250 nm. At high density, separation is 300 to 350 nm. At medium density, separation is 400 nm to 500 nm. At low density, separation is 500 nm or more. In some implementations (for example, some low density implementations) 2-dimensional patterning with photoresist is sufficient to sequester DNA nanoballs or other discrete nucleic acid samples. In some implementations (for example, some medium, high, or ultra-high density implementations), to reduce risk that discrete samples will not remain in single locations, smaller samples may be required, which may require 3-dimensional patterning for more efficient capturing of fluorescence from tagged DNA nanoballs or other tagged nucleic acid samples. In such implementations, 3-dimensional patterned well nanostructures can be developed by non-binding material as a well wall and binding material for the well bottom surface for sequestering DNA nanoballs.

FIG. 1 shows an example of a flow cell 10 usable in a sequencing system. The flow cell 10 includes a substrate 12 and a cover 14. The substrate 12 has an inner substrate surface 16 and the cover has an inner cover surface 18 facing the inner substrate surface 16, with surfaces 16, 18 spaced apart by a height h to define a fluid gap 20 between them.

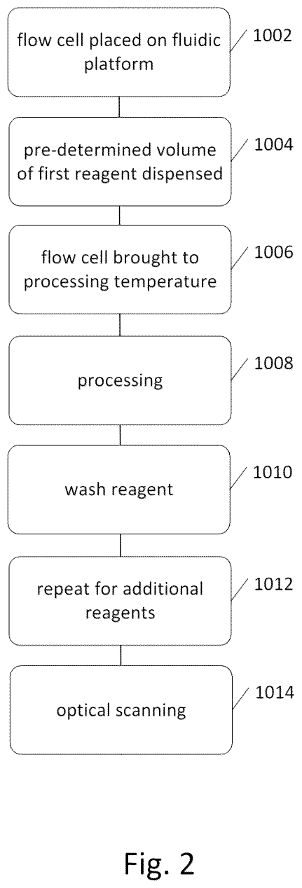

FIG. 2 shows an example process flow for the flow cell 10 of FIG. 1, which in this example is part of a genetic sequencing process flow. At step 1002, the flow cell 10 is placed on a fluidic platform. Subsequently, at step 1004, a predetermined volume of a first reagent is dispensed through the fluid inlet 22 of substrate 12. Subsequently, at step 1006, the flow cell is ramped to a processing temperature, and at step 1008, the flow cell is maintained at the processing temperature for a given amount of time. Subsequently, at step 1010, fluid inlet 22 and outlets 24 facilitate introduction of a buffer fluid and washing of the reagent from the flow cell 10. Subsequently, at step 1012, steps 1004-1010 may be repeated for as many additional reagent and washing cycles as required for the analysis being carried out. Subsequently, at step 1014, a reagent to facilitate optical scanning may be dispensed into the flow cell 10 and flow cell 10 may be optically scanned. Depending on the configuration of the system, optical scanning may take place on the fluidic platform, or the flow cell 10 may be transported to a separate optical scanning platform.

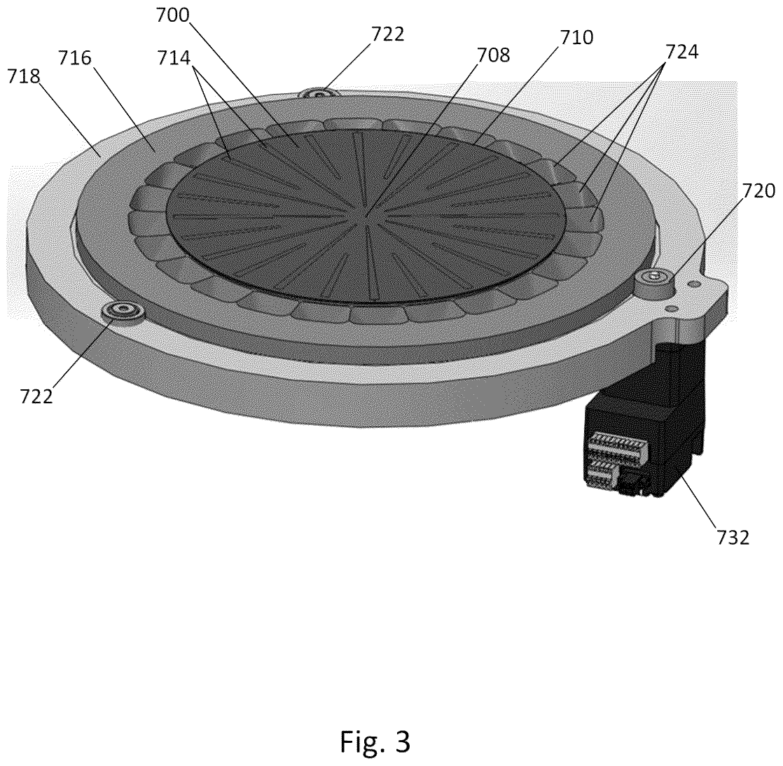

### Frameless Flow Cell—FIGS. 3-6

In the flow cell 10 shown in FIG. 1, fluid enters and exits through a discrete fluid inlet 22 and a discrete fluid outlet 24, which are tunnels formed through substrate 12. The outer perimeter of the flow cell is completely surrounded by a frame 28. Frame 28 may be an adhesive of uniform height securing the substrate 12 and cover 18 and extending unbroken about the perimeter of flow cell 10. FIGS. 3-6 show an example of a frameless flow cell system in which the outer perimeter of the flow cell is open. Leaving the perimeter open and without a frame to limit flow to discrete inlets and outlets helps to reduce resistance to flow, allowing for minimization of the flow gap between the flow cell's cover and substrate.

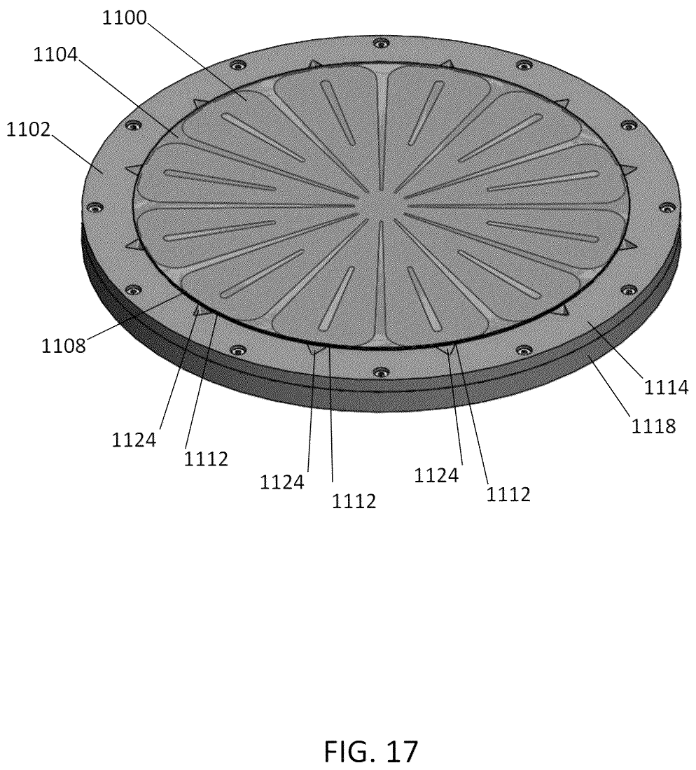

In the example of FIGS. 3-6, the flow cell is “frameless” with a completely open outer perimeter, with no adhesive or other framing present and the outer edges of the flow cell substrate and cover. In other examples (including the example of FIGS. 17-25 discussed below), a flow cell may be “frameless” while still having some discrete regions of adhesive or other framing present at the outer edges of the flow cell substrate and cover. In both the example of FIGS. 3-6 and the example of FIGS. 17-25, the flow cells are “frameless” in that the gap between the flow cell substrate and cover act as the fluid outlet for the flow cell, as opposed to for instance the discrete tunnel formed through the flow cell substrate in FIG. 1.

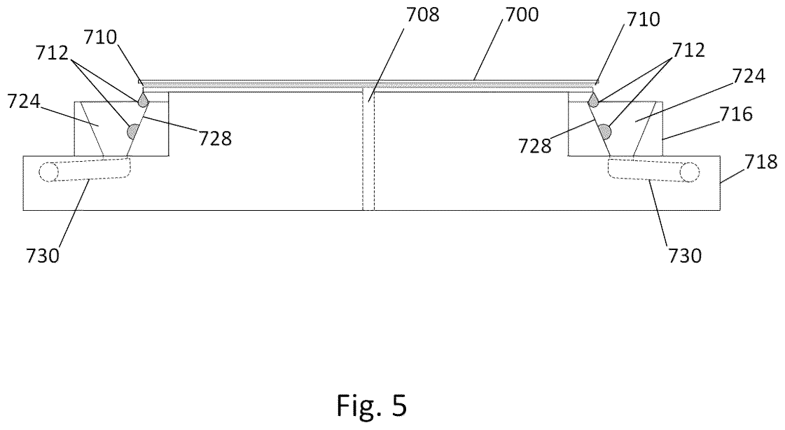

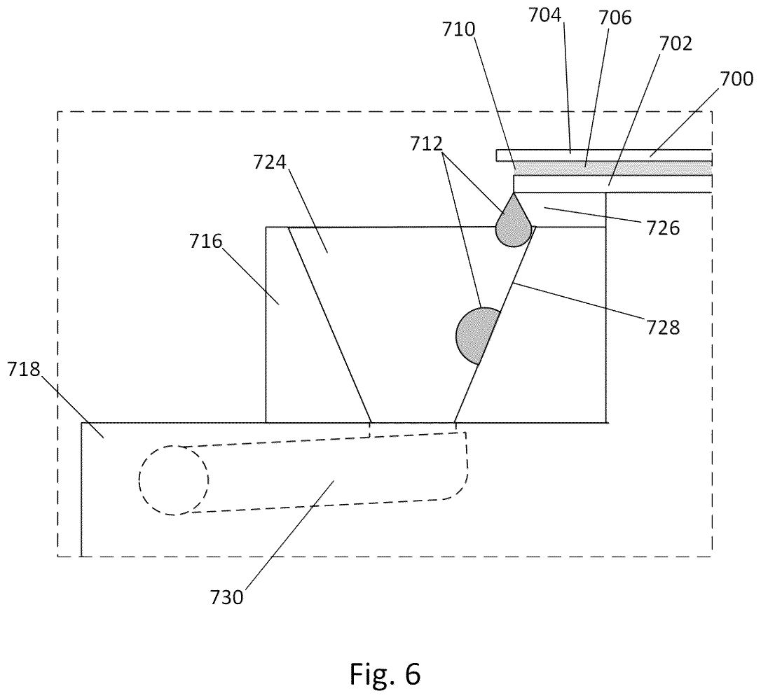

The flow cell system of FIGS. 3-6 includes a flow cell with a substrate 702 (see FIG. 6) configured to support an analyte array (e.g. via discrete attachment sites similar to the attachment sites 26 on the substrate 12 shown in FIG. 1) and a cover 704 spaced from the substrate 702. As shown most clearly in FIG. 6, the substrate 702 and cover 704 define a fluid gap 706 between the substrate 702 and cover 704. Fluid enters the flow cell of FIGS. 3-6 by an inlet 708 in fluid communication with the fluid gap 706 (see FIG. 5) and exits the flow cell at its outer perimeter 710 (see FIG. 6). The flow cell of FIGS. 3-6 is configured such that fluid enters the flow cell at the inlet 708, flows through the fluid gap 706, and exits the flow cell at the outer perimeter 710 such that the fluid drops down from the outer perimeter 710 as droplets 712.

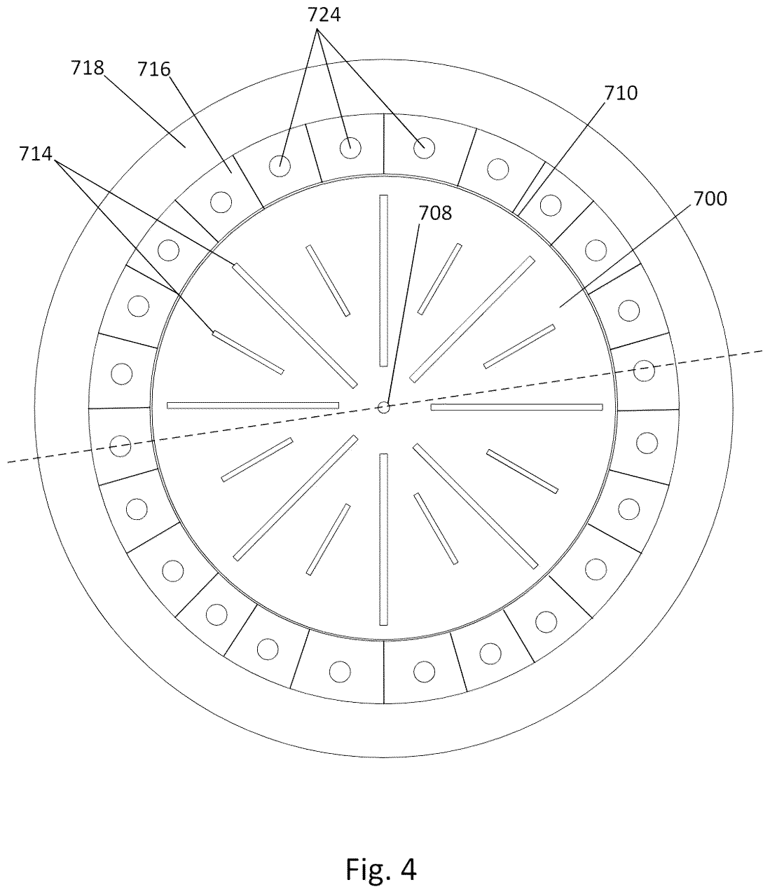

In this example, spacers 714 (see FIGS. 3, 4) space the substrate 702 and cover 704 apart to define the fluid gap 706 while leaving the outer perimeter 710 open and frameless. In this particular example, the spacers 714 are generally aligned towards a central area of the flow cell and extend radially from the central area where the inlet 708 is located. In this particular example, outer portions of the spacers 714 are inset relative to the outer perimeter 710 of the flow cell. Spacers 714 may be a patterned adhesive applied to the substrate 702 or any other structure or material sufficient to create a defined spacing between the substrate 702 and cover 704 while leaving the outer perimeter 710 of the flow cell open and frameless.

The flow cell system shown in FIGS. 3-6 includes a flow cell 700, which is mounted on top of a funnel disk 716, which is mounted on top of a fluidic channel fixture 718 in a rotatable fashion. Drive wheel 720 (actuatable by drive motor 732) can rotate the funnel disk 716 and flow cell 700 relative to the fluidic channel fixture 718. Roller bearings 722 maintain centering of the funnel disk 716 and flow cell 700 on the fluidic channel fixture 718 during rotation.

The funnel disk 716 includes a series of funnels 724 that are positioned below the outer perimeter 710 of the flow cell 700 and configured to collect droplets 712 that drop down from the outer perimeter 710 of the flow cell 700. In other implementations, the series of funnels 724 may be replaced with a single funnel or other mechanism for collecting droplets 712 from the outer perimeter 710 of the flow cell 700.

As shown in FIG. 6, the collection funnels 724 are vertically spaced from the flow cell 700 by a drop gap 726. More specifically, the tops of the collection funnels 724 are vertically spaced from the bottom of the flow cell 700 substrate 702 by a drop gap 726. In some implementations, drop gap 726 encourages formation of droplets 712 as the fluid flows out from the outer perimeter 710. In some implementations, drop gap 726 also ensures that fluid flowing out from the outer perimeter 710 will be collected in the funnels 724 (e.g. as opposed to being drawn into the interface between the flow cell 700 and the funnel disk 716 via capillary action).

As also shown in FIG. 6, the collection funnels 724 include a sloped wall 728 configured to direct the collected droplets 712 to a collection channel 730 of the fluidic channel fixture 718. In this particular example, the sloped wall 728 has a slope that is greater than 40 degrees (relative to horizontal) to help overcome surface tension of the droplets 712 so that they will move into collection channel 730. In some implementations, the sloped walls 728 of funnels 724 may be sloped at angles relative to horizontal of greater than 35 degrees, greater than 50 degrees, or greater than 60 degrees.

In the particular example shown in FIGS. 3-6, the collection channels 730 are also sloped relative to the horizontal and away from its associated funnel 724. In some implementations, the collection channels 730 may be sloped by at least five degrees relative to horizontal, by seven degrees relative to horizontal, or by greater amounts. This slope will facilitate movement of one or more droplets along the channels 730 away from the funnels 724. The collection channels 730 may also be sized to encourage flow of droplets (or pooled droplets) along the channels 730. For example, the collection channels may be greater than 3 mm wide, or greater than 5 mm wide, to minimize influence of channel wall surface tension on the droplets or pooled droplets.

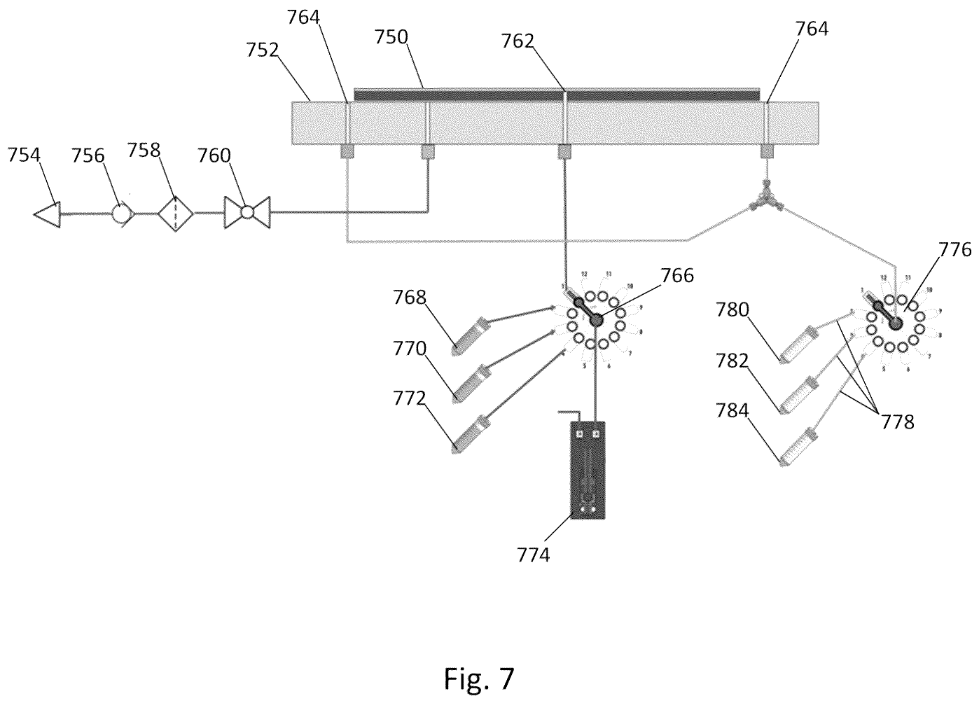

### Fluid Recycling—FIGS. 7-9

In at least some of the implementations of flow cell systems described herein, it may be desirable to collect fluid exiting a flow cell such that some of the collected fluid is directed into a waste pathway while some of the collected fluid is instead directed into one or more reagent recycling pathways so that at least some of the collected fluid (e.g. reagent) can be used again, either in continued analysis of the same flow cell or in analyses of other flow cells.

### FIG. 7

FIG. 7 shows an example of a flow cell system in which collected fluid may be directed along different pathways depending on the state of the system. The system includes a flow cell 750 mounted on a vacuum chuck 752. Flow cell 750 is held on vacuum chuck 752 by negative pressure generated by a vacuum sub-system including a vacuum pump 754, one way valve 756, filter 758, and two way valve 760.

Fluid is introduced into the flow cell 750 at inlet 762 and collected from the flow cell 750 at outlets 764. Different fluids may be selectively introduced into the flow cell 750 via inlet 762 by a selector valve 766. Selector valve 766 may be cycled through different states to fluidically connect sources of a first type of reagent 768, a second type of reagent 770, and a wash fluid 772. Fluid may be directed from the connected source 768, 770, or 772 into the flow cell 750 via inlet 762 by a syringe pump 774 or other suitable pumping mechanism.

In the example of FIG. 7, a second selector valve 776 facilitates selectively collecting fluids exiting the flow cell 750 into different collection channels 778 and receptacles 780, 782, 784. More specifically, the system may be configured to cycle selector valve 776 between a state where collected fluid is directed along a channel 778 in fluid communication with a collection receptacle 780 for a first type of reagent, a state where collected fluid is directed along a channel 778 in fluid communication with a collection receptacle 782 for a second type of reagent, and a state where collected fluid is directed along a channel 778 in fluid communication with a collection receptacle 784 for waste fluid. For instance, in one example implementation of the system shown in FIG. 7, the system may be configured such that when the selector valve 766 is in a state to direct fluid from the source of the first type of reagent 768 into the flow cell 750, the selector valve 776 is in a state to direct fluid collected from the flow cell 750 into the collection receptacle for the first type of the reagent 780. Similarly, the system may be configured such that when the selector valve 766 is in a state to direct fluid from the source of second type of reagent 770 into the flow cell 750, the selector valve 776 is in a state to direct fluid collected from the flow cell 750 into the collection receptacle for the second type of the reagent 782. Similarly, the system may be configured such that when the selector valve 766 is in a state to direct fluid from the wash fluid source 772 into the flow cell 750, the selector valve 776 is in a state to direct fluid collected from the flow cell 750 into the waste collection receptacle 784.

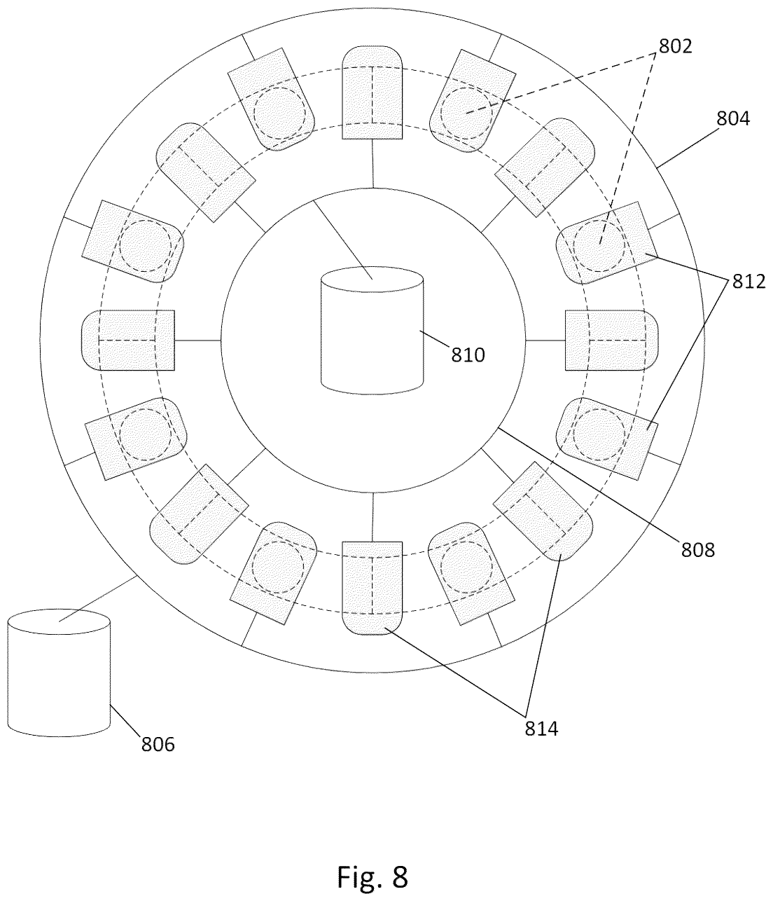

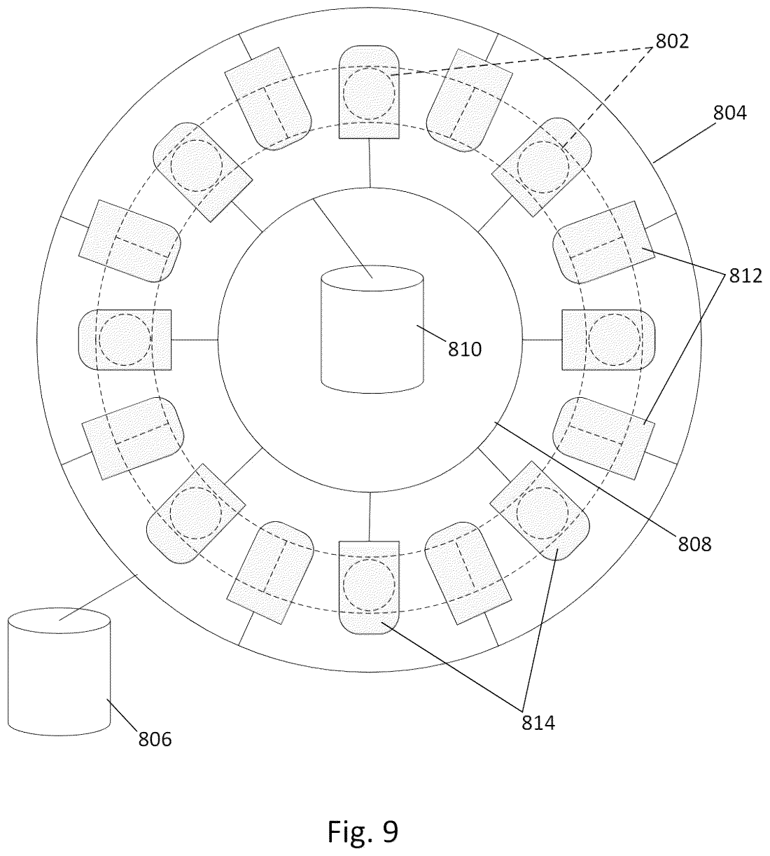

### FIGS. 8-9

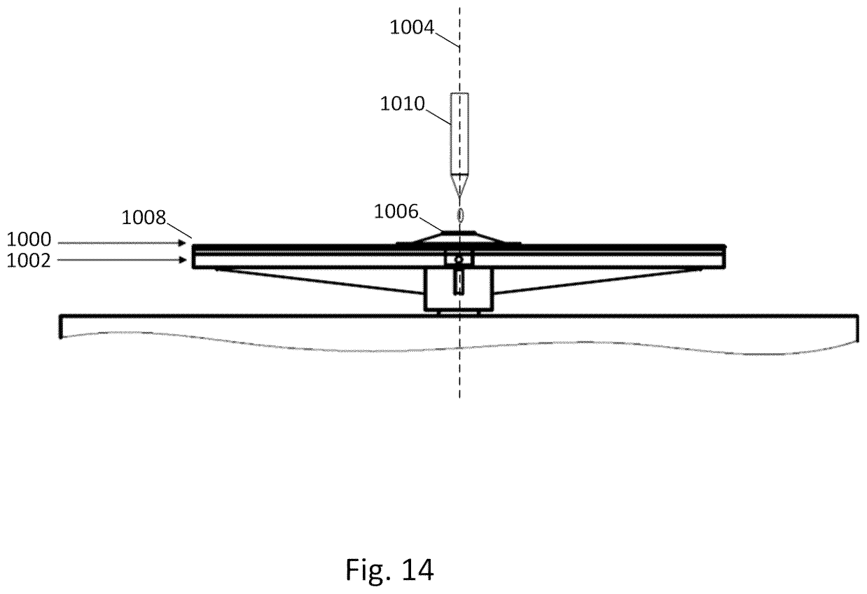

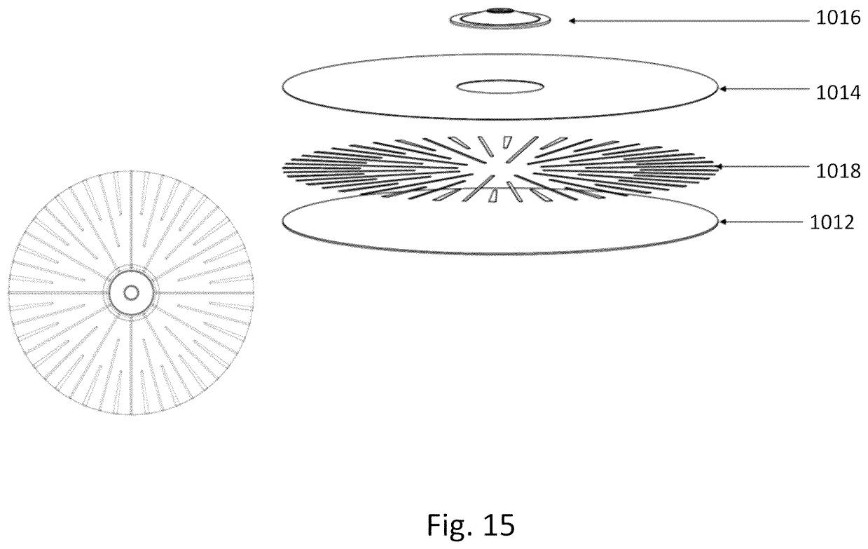

FIGS. 8-9 schematically shows another example of a flow cell system that is actuatable between two states in which collected fluid from a flow cell can be directed along two different pathways depending on the state of the system. This example includes a funnel disk with a series of funnels 802 similar to the funnel disk 716 and funnels 724 shown in FIGS. 9-12. In FIGS. 8-9, the funnel disk and funnels 802 are shown in dashed lines, with the dashed circles indicating outlets of the funnels 802. This example also includes a fluidic channel fixture similar to the fluidic channel fixture 718 of FIGS. 3-6. The fluidic channel fixture in the example of FIGS. 14 and 15 includes two different fluid pathways, a first pathway 804 that is fluidically connected to a first collection receptacle 806, and a second pathway 808 that is fluidically connected to a second collection receptacle 810.

Similar to the system of FIGS. 3-6, the system of FIGS. 8-9 is configured to rotate the funnel disk including funnels 802 relative to the fluidic channel fixture including fluidic pathways 804, 806. When the system is actuated to a first state shown in FIG. 8, the funnels 802 are configured to direct collected droplets from a flow cell (not shown) along the first pathway 804 to first collection receptacle 806, which may be, for example, a waste receptacle. More specifically, when in the first state shown in FIG. 8, the funnels 802 are positioned over and in fluid communication with collection channels 812 of the first pathway 804 so that droplets will drop down through funnels 802 and into the collection channels 812 (in a similar fashion to what is shown in FIG. 6) and enter the first pathway 804 to the first collection receptacle 806.

When the system is actuated to a second state shown in FIG. 9, the funnel disk and its funnels 802 are repositioned relative to the fluidic channel fixture and its fluidic pathways 804, 808, such that the funnels 802 are configured to direct collected droplets from the flow cell along the second pathway 808 to second collection receptacle 810, which may be, for example, a reagent fluid recycling receptacle. More specifically, when in the second state shown in FIG. 9, the funnels 802 are positioned over and in fluid communication with collection channels 814 of the second pathway 808 so that droplets will drop down through funnels 802 and into the collection channels 814 and enter the second pathway 808 to the second collection receptable 810.

In other implementations, a fluidic channel fixture similar to what of FIGS. 8-9 may include additional fluid pathways so that collected droplets may be directed to three or more different collection receptacles.

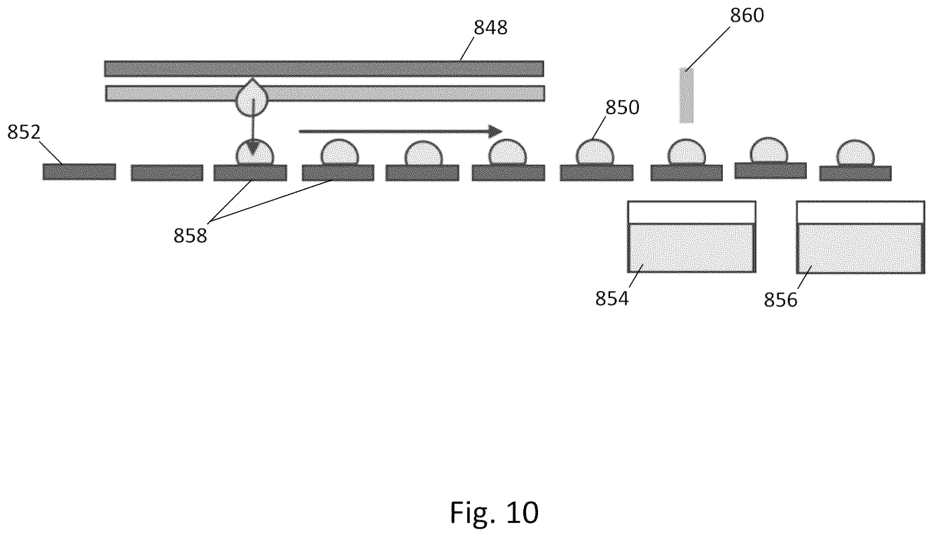

### FIG. 10

FIG. 10 shows another example of a recycling sub-system that may be incorporated into a flow cell system, including the various flow cell systems described herein.

In this example, the system is configured to sort droplets collected from a flow cell 848 into different groups. More specifically, in this example, the system is configured to sort droplets 850 from a common droplet path 852 into different paths including, for instance, a recycling droplet path represented by recycling receptacle 854 and waste droplet path represented by waste receptacle 856. In other implementations, additional paths and receptacles may be incorporated into the sorting system (e.g. one path and receptacle for a first type of reagent, additional paths and receptacles for additional types of reagents, and an additional path and receptacle for waste fluid).

In the example of FIG. 10, the common droplet path 852 is a drop conveyor including a series of moving pads 858, each pad 858 configured to collect an individual drop 850 as it exits flow cell 848 and selectively hold the droplet 850 thereon as it moves along the conveyor. For instance, the pads 858 may include dielectric surfaces covering high voltage electrical pads allowing electrostatic manipulation of individual drops (e.g. selectively retaining or releasing the drop from the pad 858 depending on the state of the high voltage electrical pad therein). In the example of FIG. 10, a trace dye detector 860 or other type of sensor interrogates each drop as moves along the conveyor to provide data to the system that is used to determine which receptacle 854, 856 the droplet 850 should be sorted into. In other implementations, it may not be necessary to individually interrogate each droplet to determine which path it should be directed to, and sorting may be accomplished in other ways (e.g. during certain times or operating states of the system, collected droplets may be directed to the waste receptacle while at other times and operating states, collected droplets may be directed to one or more reagent recycling receptacles).

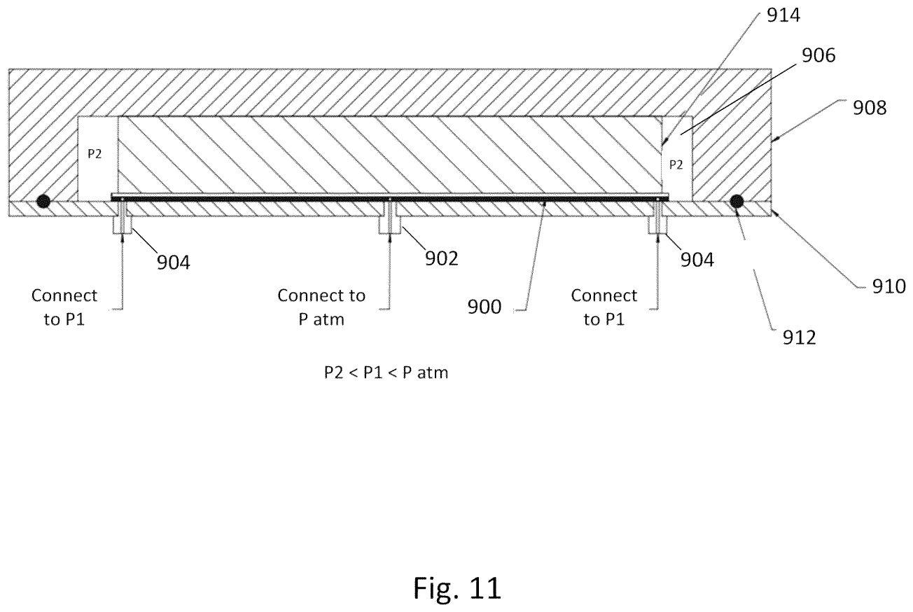

### Frameless Flow Cell—Vacuum Chamber—FIGS. 11-13

FIGS. 11-13 show examples of flow cell systems that are configured for use in conjunction with a vacuum sub-system that generates a reduced pressure at the outlet of the flow cell (including when the “outlet” of the flow cell is the frameless outer perimeter of the flow cell) to reduce resistance to fluid flow through the flow cell's fluid gap.

The example in FIG. 11 includes a flow cell 900 that, although not shown in detail, includes a substrate configured to support an analyte array and a cover spaced from the substrate to define a fluid gap between the substrate and the cover. The flow cell 900 also includes an inlet 902 in fluid communication with the fluid gap and outlets 904 in fluid communication with the fluid gap. In the particular example shown, the inlet 902 extends through the substrate of the flow cell 900 near or at the middle of the flow cell 900 and the outlets 904 extend through the substrate of the flow cell 900 near or at the outer perimeter of the flow cell 900.

A vacuum sub-system (not shown) generates reduced pressure at the outlets 904 in order the reduce resistance to fluid flow through the fluid gap of the flow cell 900. In one implementation, pressure at both the fluid inlet 902 and the fluid outlets 904 is regulated, with fluid inlet 902 connected to a pressure that is higher than the pressure that fluid outlets 904 are connected to. For example, fluid inlet 902 may be connected to atmospheric pressure (or an approximate thereof) and fluid outlets 904 may be connected to a pressure that is below atmospheric pressure.

In the example of FIG. 11, flow cell 900 is positioned inside a vacuum chamber 906. The vacuum sub-system may be further configured to regulate pressure inside the vacuum chamber 906 to generate a reduced pressure inside the chamber, such that a reduced pressure is applied to an outer (top) surface of the flow cell 900 cover. The vacuum chamber 906 shown in FIG. 11 is vacuum chuck that includes a chuck cover 908, a chuck plate 910, and an o-ring 912 to seal the interface between the cover 908 and plate 910. An elastic member 914 inside the vacuum chamber 906 helps to hold the flow cell 900 in position inside the vacuum chamber 906.

In some implementations, the reduced pressure inside the vacuum chamber 906 may help to counteract the reduced pressure at the fluid outlets 904 of the flow cell 900, reducing the possibility that the flow cell 900 cover could collapse during flow cell filling.

In some implementations of the system shown in FIG. 11, during operation, the vacuum sub-system (e.g. one or more vacuum pumps configured to regulate pressure at the fluid inlet 902, outlet 904, and in the interior of the vacuum chamber 906) regulates pressures such that pressure at the fluid inlet 902 of flow cell 900 is greater than pressure in the interior of vacuum chamber 906 (which acts on outer surface of the flow cell 900 cover), which is greater than pressure at the fluid outlets 904 of flow cell 900.

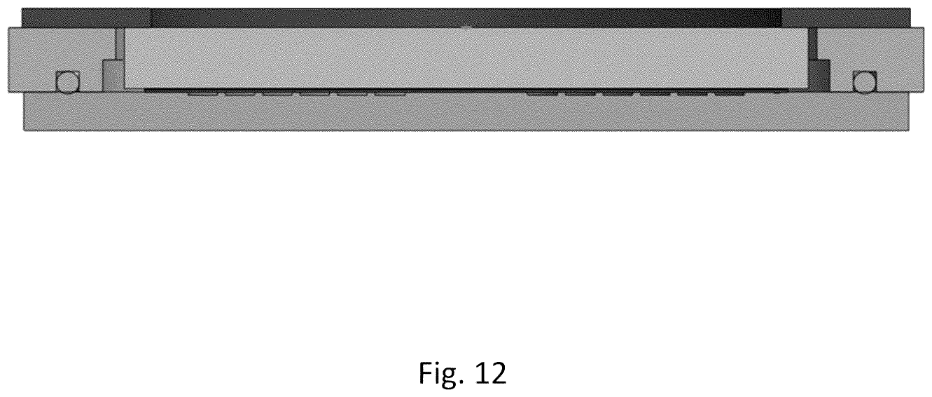

FIG. 12 shows another example of a flow cell inside a vacuum chamber.

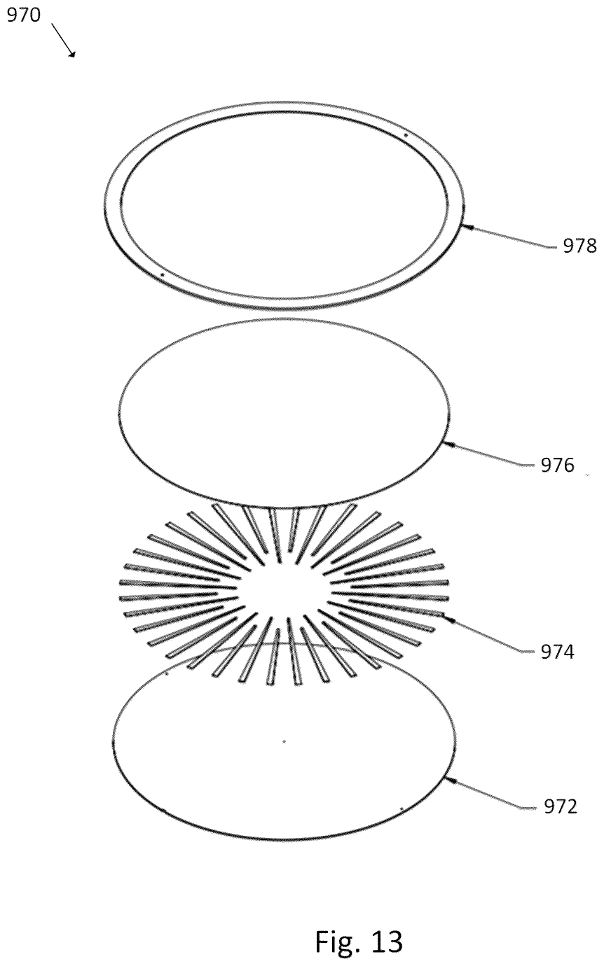

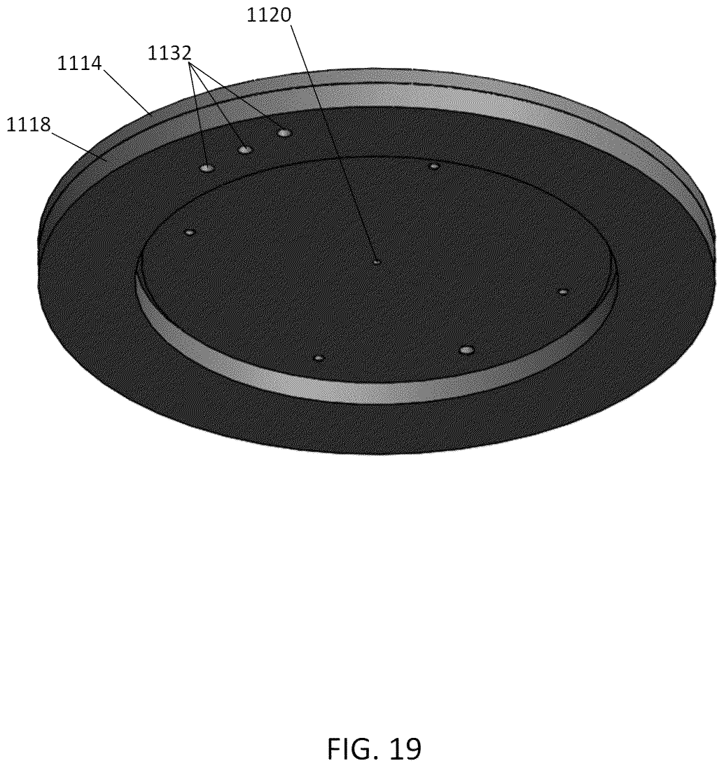

FIG. 13 shows (in an exploded view) an example of a flow cell 970 in which the flow cell 970 includes an open outer perimeter that acts as the flow cell's 970 outlet. The flow cell 970 in FIG. 19 includes a substrate 972, spacers 974, a cover 976, and a frame 978. Flow cell 970 is usable with a vacuum sub-system configured to generate a reduced pressure at the open outer perimeter of the flow cell 970 in order to reduce resistance to fluid flow through a fluid gap between the substrate 972 and cover 976.

Flow cell 970 may be positioned inside a vacuum chamber similar to those shown in FIGS. 11 and 12. The vacuum sub-system may be configured to generate reduced pressures at the outer perimeter of the flow cell 970 and at the outer surface of the cover 976. In one implementation similar to that shown in FIG. 11, the vacuum sub-system may be configured to generate reduced pressures such that pressure at a fluid inlet of the flow cell 970 is greater than pressure at the outer perimeter of the flow cell 970, which is greater than pressure at the outer surface of the cover 976 of the flow cell.

### Frameless Flow Cell—Spin Coating—FIGS. 14-16

FIGS. 14-16 show examples of flow cell systems and flow cells for use in such systems in which the flow cell is spun in order to move fluid from a central inlet in the flow cell to its circumference where the fluid exits the flow cell. Rotational speed of the flow cell may depend on the fluid resistance and force to push fluid to the edge.

Similar to some of the flow cells in earlier described examples, the flow cells of the examples shown in FIGS. 14-16 may have outer perimeters that are open or frameless. Similar to some of the earlier examples, the flow cells in these examples may include a substrate configured to support an analyte array, a cover spaced from the substrate to define a fluid gap between the cover and substrate, an inlet in fluid communication with the fluid gap, and an outer perimeter of the flow cell. Systems using the flow cells of FIGS. 14-16 may be configured such that fluid enters the flow cell at the inlet, flows through the fluid gap, and exits the flow cell at the outer perimeter such that the fluid drops down from the outer perimeter as droplets.

In the example of FIG. 14, the flow cell 1000 is mounted on an actuator 1002, which in this particular example is a rotary chuck. The actuator 1002 is configured to spin the flow cell about a vertical axis 1004 to facilitate flow from an inlet 1006 of the flow cell 1000 to its outer perimeter 1008. In the example shown in FIG. 14, the inlet 1006 is a centrally located opening extending through a cover of the flow cell 1000 into which fluid may be dispensed by dispenser 1010.

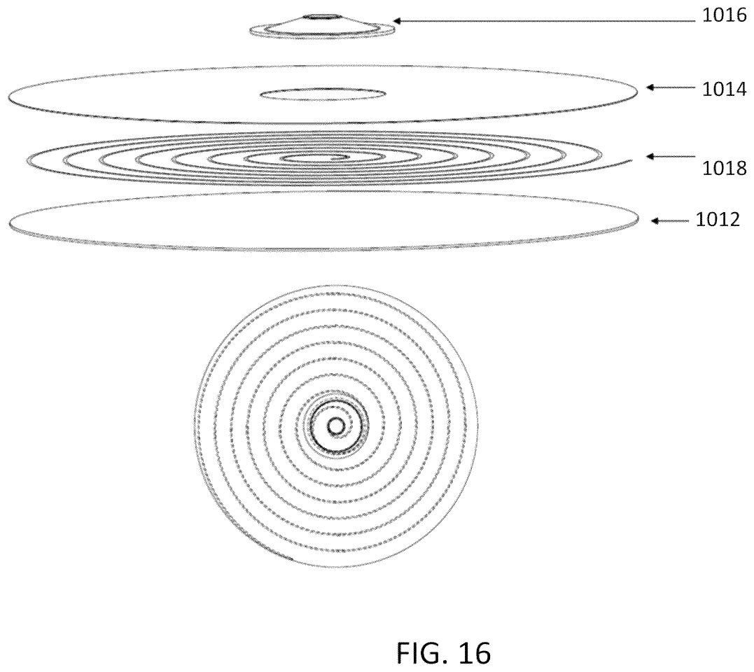

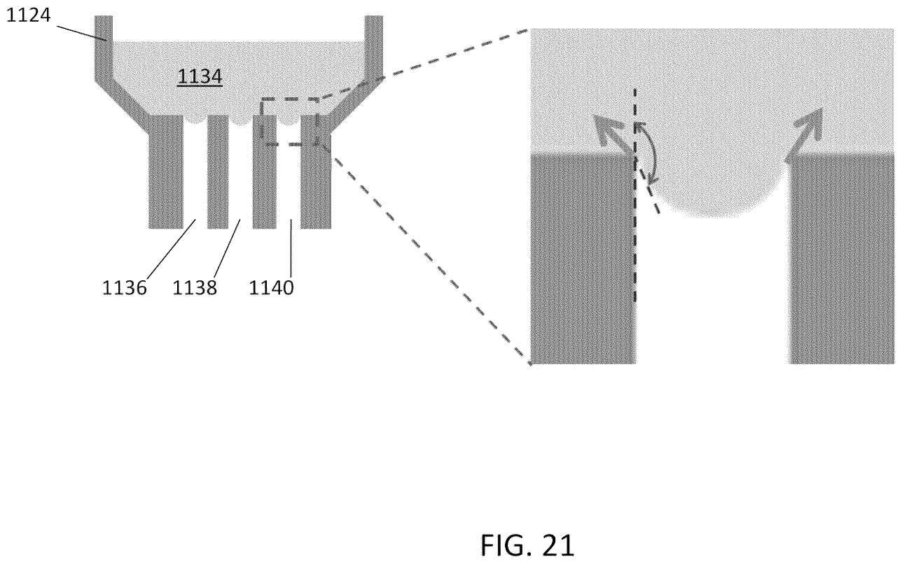

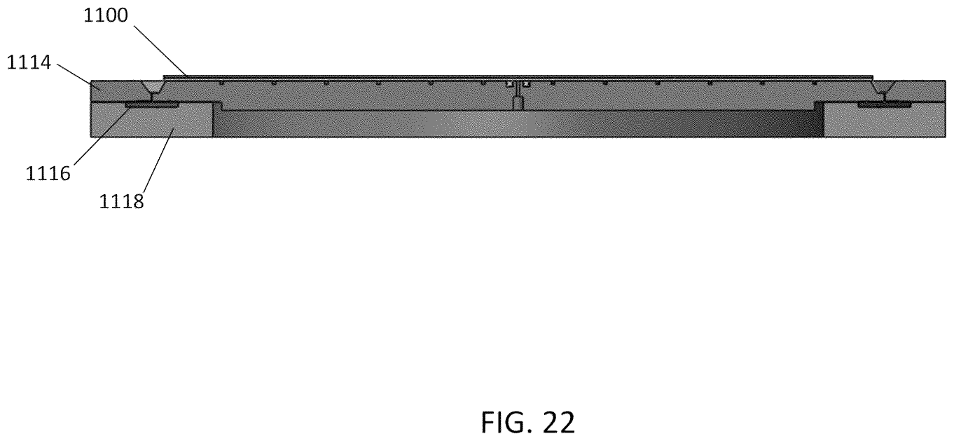

FIGS. 15 and 16 show examples of flow cells usable with the actuator 1002 of FIG. 14. Both examples include a substrate 1012, cover 1014, and inlet 1016 mounted over an opening in cover 1014. FIGS. 21 and 22 differ in the configuration of spacers 1018 between the substrate 1012 and cover 1014. In the example of FIG. 15 the spacers 1018 are arranged in a radially extending pattern. In the example of FIG. 16 the spacers 1018 are arranged in a spiral pattern.

Frameless Flow Cell with Partially Open Outer Perimeter—FIGS. 17-25

FIGS. 17-25 show another example of a flow cell system. In this example the flow cell system includes a flow cell 1100 positioned on a fluid collector 1102.

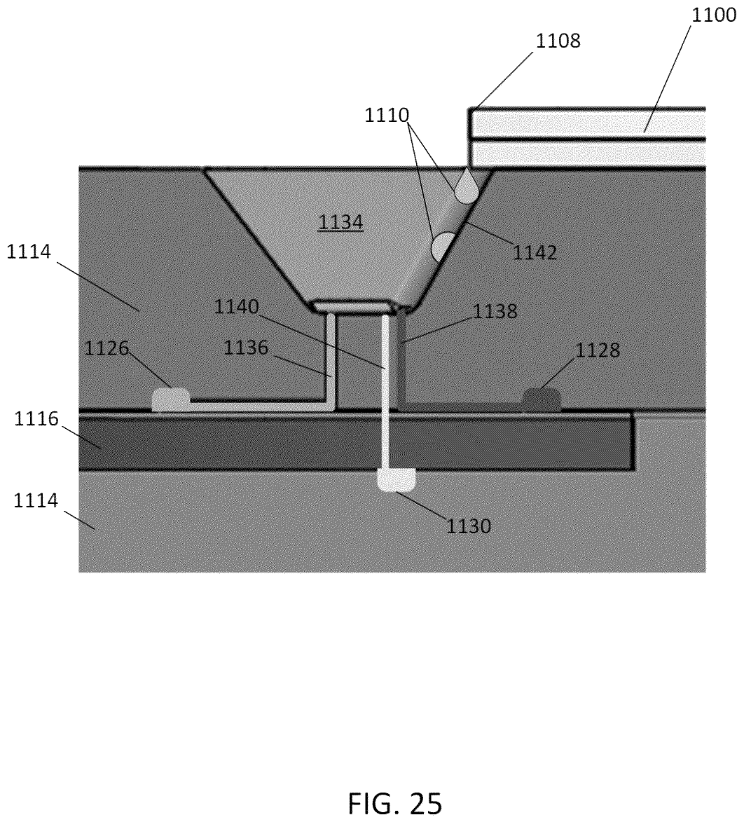

FIGS. 17-25 do not show the flow cell 1100 in detail, as it has several similarities to flow cells described in earlier examples. For instance, the flow cell 1100 of FIGS. 17-25 includes a substrate configured to support an analyte array and a cover spaced from the substrate to define a fluid gap between them. A spacer (in this example, a pattern of adhesive 1104) spaces the cover from the substrate. The flow cell 1100 also includes an inlet in fluid communication with the fluid gap and an outer perimeter (1108 in FIGS. 17 and 25).

Similar to the flow cells of earlier examples, in the example of FIGS. 17-25 fluid enters the flow cell at the inlet 1120, flows through the fluid gap, and exits the flow cell at the outer perimeter 1108 between the substrate and the cover. As shown in FIG. 25, fluid exiting the flow cell drops down from the outer perimeter 1108 as droplets 1110.

Also similar to the flow cells of earlier examples (such as the flow cells in the examples of FIGS. 3-6, FIG. 13, FIG. 14, FIG. 15, and FIG. 16), the flow cell 1100 of FIGS. 17-25 is frameless. The flow cell 1100 does not have a frame that entirely surrounds its outer perimeter 1108.

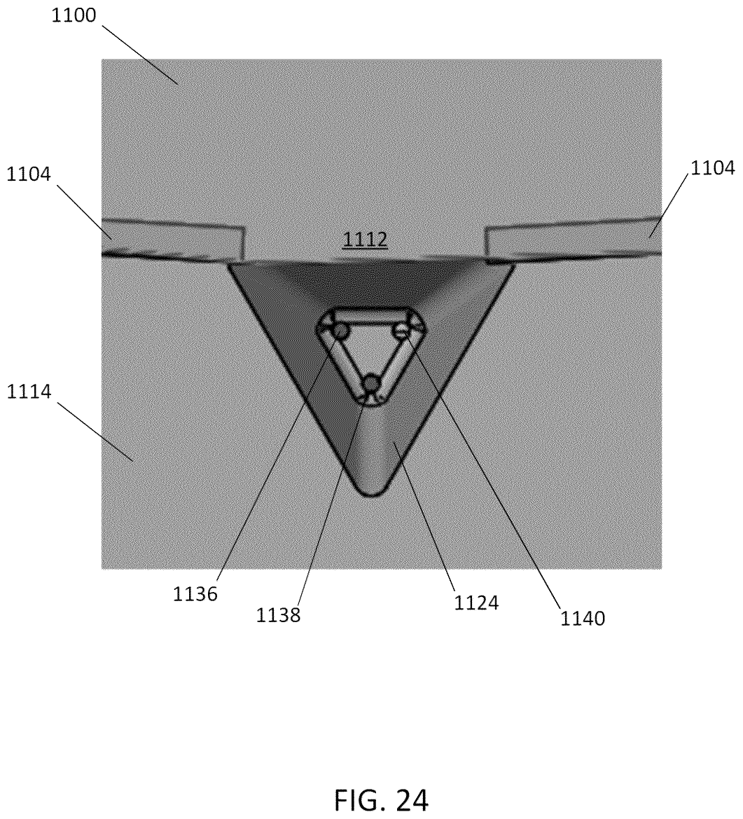

Unlike some of the earlier examples, however, the outer perimeter 1108 of flow cell 1100 is only partially open. Compare, for instance, the flow cell 700 of FIGS. 3-6 to the flow cell 1100 of FIGS. 17-25. The flow cell 700 of FIGS. 3-6 has adhesive spacers 714 that are located entirely in the interior of the flow cell 700 and do not extend all of the way to its outer perimeter 710. The outer perimeter 710 of the flow cell 700 is thus completely open. In contrast, the flow cell 1100 of FIGS. 17-25 has adhesive spacers 1104 that do extend all of the way to and partially around the outer perimeter 1108, although gaps in the adhesive 1112 (see FIGS. 17 and 24) still make the outer perimeter 1108 partially open.

In certain implementations, the number and dimensions of gaps in the adhesive 1112 at the outer perimeter 1108 may be selected such that the outer perimeter is at least 2.5% open, at least 5% open, at least 10% open, or a greater percentage. In the particular example shown in FIGS. 17-25, there are twelve adhesive gaps 1112 at the outer perimeter 1108 of the flow cell 1100. In other implementations, there may be at least five separate adhesive gaps at the outer perimeter, in other implementations, there may be at least ten separate adhesive gaps at the outer perimeter.

In certain implementations, an open or at least partially open outer perimeter of a flow cell can reduce resistance to fluid flow through the flow cell's fluid gap, which may allow for cycling fluids through relatively large flow cells with relatively minimal fluid gaps at reasonably fast fluid cycling rates. In some implementations, the flow cell substrate may have a surface area of at least 150 cm² and a fluid gap volume that is less than 1.5 mL. In some implementations, the flow cell substrate may have a surface area of at least 225 cm² and a fluid gap volume that is less than 1 mL. In some implementations, the flow cell substrate may have a surface area of at least 300 cm² and a fluid gap volume that is less than 0.75 mL.

### Fluid Recycling—FIGS. 17-25

The flow cell system of FIGS. 17-25 is actuatable between several states such that the system can direct fluid exiting the flow cell along different fluid pathways. In the specific example shown, the system is configured to alternatively flow a first type of reagent fluid, a second type of reagent fluid, and a wash fluid through the flow cell. In the specific example shown, the system includes three different fluid pathways along which fluid exiting the flow cell can be directed: a waste pathway, a first recycling pathway, and a second recycling pathway. The system is configured such that the first type of reagent fluid will be directed along the first recycling pathway, such that the second type of reagent fluid will be directed along the second recycling pathway, and such that the wash fluid and wash fluid/reagent mixtures will be directed along the waste pathway. Other configurations accounting for other types of fluids and other numbers of fluid pathways are also possible and envisioned, depending on the types of reagents required for the particular analytic process. For instance, in other implementation, a system could utilize one type of reagent and one wash fluid, and there may be just two fluid pathways for receiving the fluids after exiting the flow cell. In another example implementation, a system could utilize four types of reagents and one wash fluid, and there may be five separate fluid pathways for receiving fluids after exiting the flow cell.

FIGS. 17-20 shows an example of a flow cell/fixture assembly of the flow cell system, in both assembled and exploded views from the top and the bottom. In this example, the assembly includes flow cell 1100, vacuum chuck 1114, gasket 1116, and bottom ring 1118.

The vacuum chuck 1116 includes an inlet 1120 sealed by O-ring 1122 for injecting fluids into flow cell 1100. The vacuum chuck 1116 also includes a common collector for collecting fluids exiting the flow cell 1100. In this particular example, the common collector is a series of collecting cups 1124 positioned about and underneath the outer perimeter of the flow cell 1100. In the particular example shown, the collecting cups 1124 are cavities extending into the body of the vacuum chuck 1116.

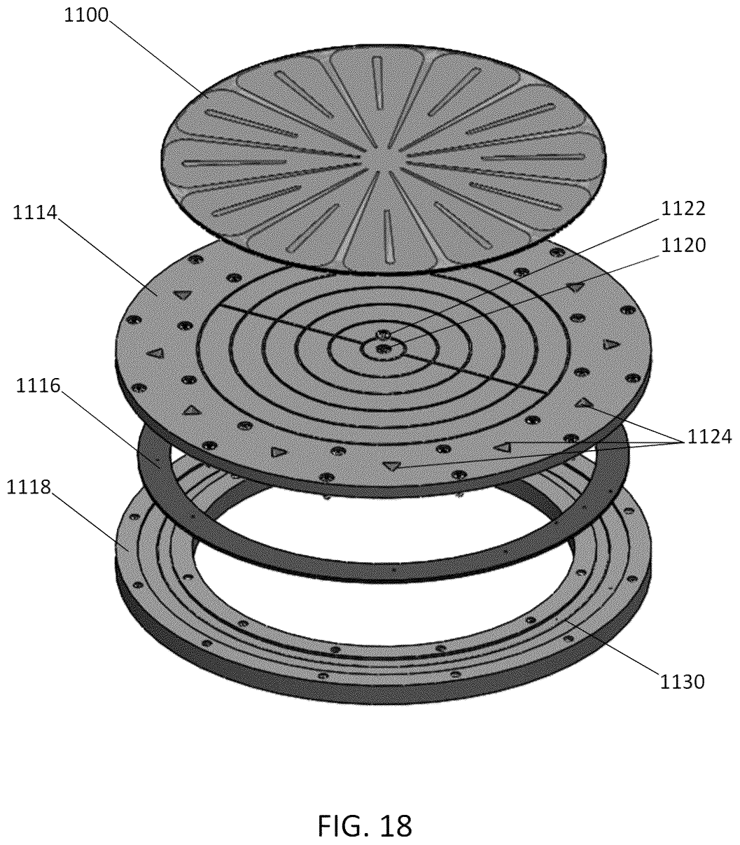

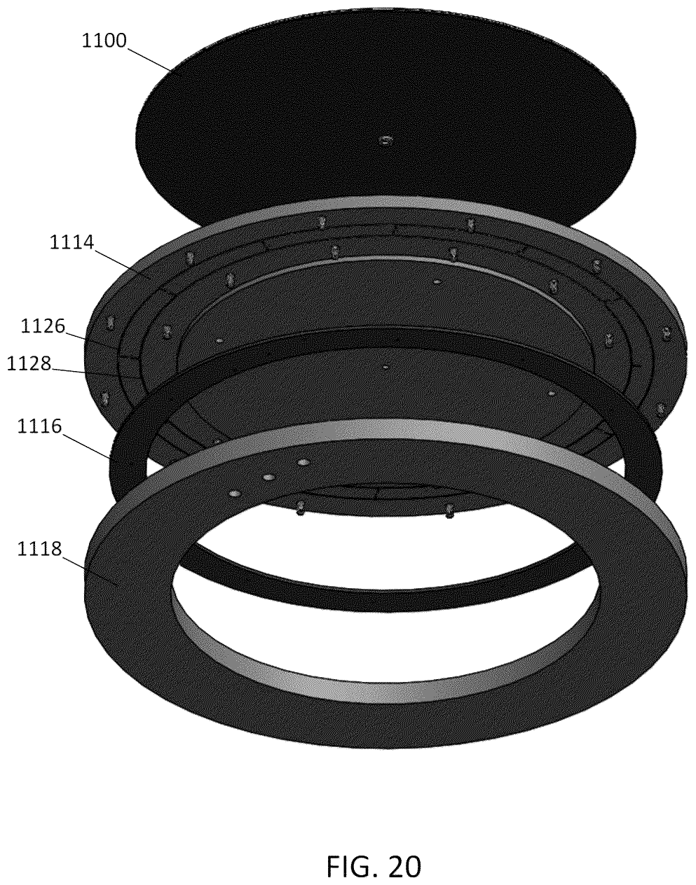

The collecting cups 1124 are each connected to three different fluid channels. Two of the fluid channels 1126, 1128 are on the underside of the vacuum chuck 1114 (see FIG. 20) and one of the fluid channels 1130 is on the top side of the bottom ring 1118 (see FIG. 18). The fluid channels 1126, 1128, 1130 are covered by gasket 1116. The fluid channels 1126, 1128, 1130 are each connected to one of the fluid outlets 1132 at the underside of bottom ring 1118 (see FIG. 19).

FIG. 21 shows a schematic cross-section of a collecting cup 1124. The system is configured such that fluid enters the collecting cup 1124 after flowing through the fluid gap in flow cell 1100. More specifically, in this example, the fluid drops down as droplets from the outer perimeter 1108 of flow cell 1100 into a collecting basin 1134 of a collecting cup 1124. A waste conduit 1136 and two recycling conduits 1138, 1140 extend from the collecting cup 1124. The waste conduit 1136 connects to the fluid channel 1126, and the waste conduits of other collecting cups also connect to the same fluid channel. The recycling conduits 1138 of each of the collecting cups 1124 are all connected to the fluid channel 1128 (along with similar recycling conduits in other collecting cups). The recycling conduits 1140 of each of the collecting cups 1124 are all connected to the fluid channel 1130 (along with similar recycling conduits in other collecting cups).

In this example, when the system is in a first state, the system is configured to draw fluid from all of the collecting cups 1124 into the waste conduits 1136 and into the common fluid channel 1126, where the fluid can subsequently be directed to waste. When the system is in the first state, fluid will not flow through the other two conduits 1138, 1140. When the system is in a second state, the system is configured to draw fluid from all of the collecting cups 1124 into the recycling conduits 1138 and into the common fluid channel 1128, where the fluid can be subsequently directed to a collection vessel for a first type of fluid to be recycled (e.g. a first type of reagent fluid). When the system is in the second state, fluid will not flow through the other two conduits 1136, 1140. When the system is in a third state, the system is configured to draw fluid from all of the collecting cups 1124 into the other recycling conduits 1140 and into the common fluid channel 1130, where the fluid can be subsequently directed to a collection vessel for a second type of fluid to be recycled (e.g. a second type of reagent fluid). When the system is in the third state, fluid will not flow through the other two conduits 1136, 1138.

In this example, the system is configured such that fluid will only drain out of the collecting basin 1134 into one of the three conduits 1136, 1138, 1140 at a time. In this particular example, the three conduits 1136, 1138, and 1140 are hydrophobic capillary openings in the bottom of collecting basin 1134 and the surface of the collecting basin is formed of a material (or treated with a coating) that renders it hydrophobic as well. In this manner, the default of the collecting cup 1124 will be that no fluid will enter any of conduits 1136, 1138, 1140 even when the collected fluid is covering all of the conduits in the bottom of basin 1134 because of the fluid's surface tension and capillary force resistance to flow.

In this example, fluid is selectively drained through one of the three conduits 1136, 1138, 1140 by applying a vacuum force to that conduit (e.g. applying a vacuum force to the common fluid channel that connects to each of those conduits in each of the collecting cups 1124). The vacuum force applied to that conduit is sufficient to overcome the inherent resistance to flow.

Vacuum force may be supplied from one or more negative pressure sources (e.g. vacuum pumps—not shown) associated with the system. The system may be configured to apply a negative pressure to the desired conduits sufficient to overcome capillary resistance to flow in those conduits such that fluid will drain out of the collecting cups 1124 only via the desired conduits and into the associated common fluid channel.

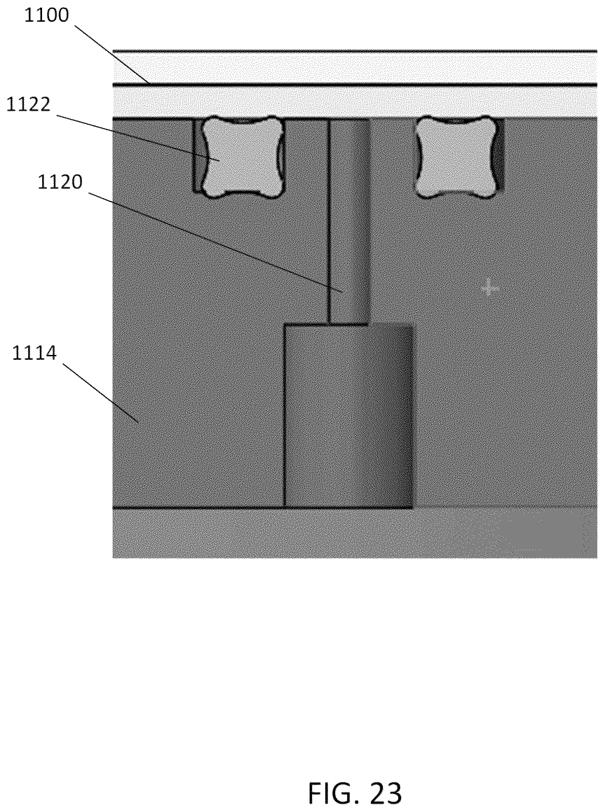

FIGS. 22-25 show additional views of the flow cell system in this example. FIG. 22 shows a cross-section of the flow cell 1100, vacuum chuck 1114, gasket 1116, and bottom ring 1118. FIG. 23 shows a close-up of the fluid inlet 1120 in the vacuum chuck 1114 and the associated o-ring 1122.

FIG. 24 shows a close-up from the top down of part of the flow cell 1100 and one of the collecting cups 1124. FIG. 25 shows a close-up cross section of part of the flow cell 1100 and the collecting cups 1124. As shown in FIGS. 24-25, the collecting cup 1124 is positioned below the outer perimeter 1108 of the flow cell 1100 and configured to collect fluid droplets 1110 that drop down from the outer perimeter 1108 of the flow cell 1100 after passing through gaps in adhesive 1112. As shown in FIG. 25, the collection cup 1124 includes a sloped sidewall 1142. The portion of the sloped sidewall 1142 immediately below the outer perimeter 1108 of the flow cell 1100 is vertically spaced from the flow cell 1100 by a drop gap. This drop gap, and the hydrophobic nature of the collection cup 1124, may help resist any tendency of droplets 1110 to enter the gap between the bottom of flow cell 1100 and the top of vacuum chuck 1114.

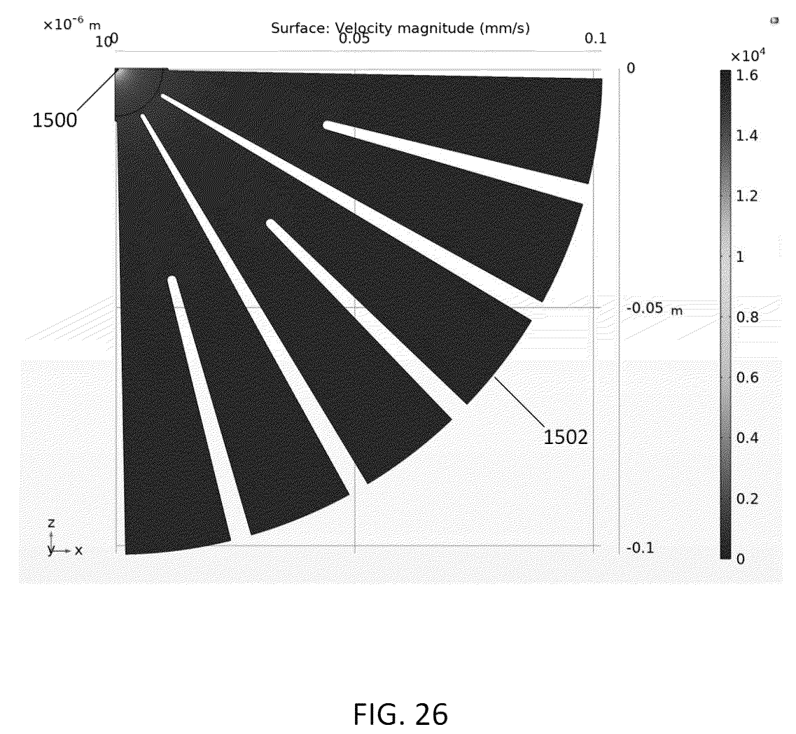

FIGS. 26-28 illustrate a simulation of filling and washing cycles for a flow cell and how the composition of the fluid exiting the flow cell changes over the course of the cycles.

FIG. 26 shows a simulated one quarter portion of a circular flow cell. Fluid enters the flow cell at fluid inlet 1500 at the center of the flow cell. The fluid outlet of the flow cell is its outer perimeter 1502, similar to the frameless flow cells described above. In this simulation, the flow cell has a fluid gap volume of approximately 0.68 mL.

In this simulation, there is one type of reagent fluid and one wash fluid. During a filling cycle, reagent is injected into the flow cell to displace the wash fluid already filling the flow cell's fluid gap. During a washing cycle, was fluid is injected into the flow cell to displace the reagent fluid.

In this simulation, an injection rate of 0.1 mL/sec is used. 15% glycerol in water is used as a surrogate for the reagent fluid and 10% glycerol in water is used as a surrogate for washing fluid. As such, the concentration change in the fluid at the flow cell's outlet can be used to model the change over from reagent to washing fluid and vice-versa and the flow cell's outlet.

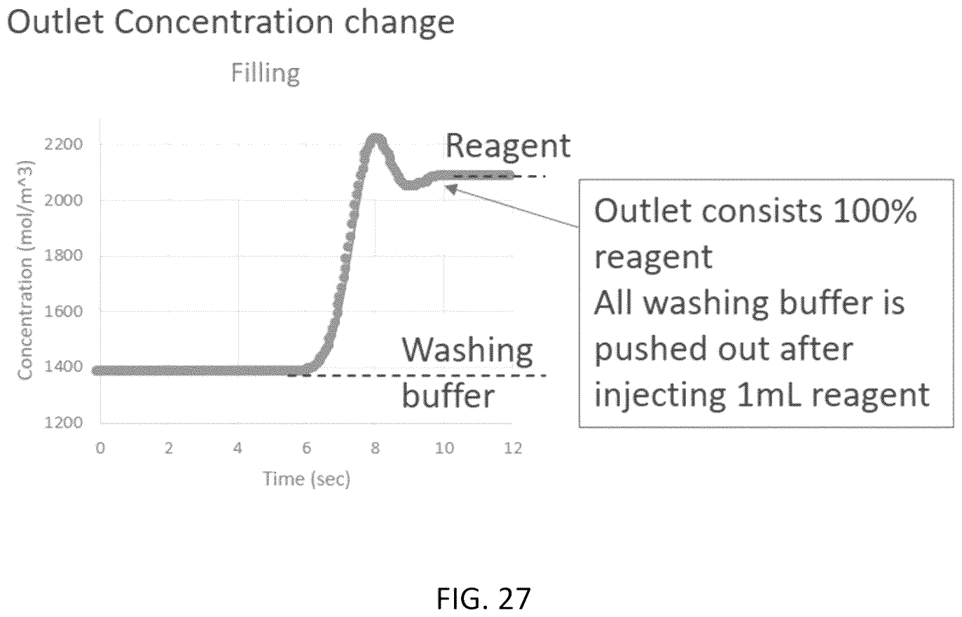

FIG. 27 shows outlet concentration change during a simulated filling cycle. FIG. 27 shows that, for this simulation, after 10 seconds of filling (injecting 1 mL of reagent surrogate) all of the washing buffer has been pushed out of the flow cell and the flow cell is entirely filled with reagent.

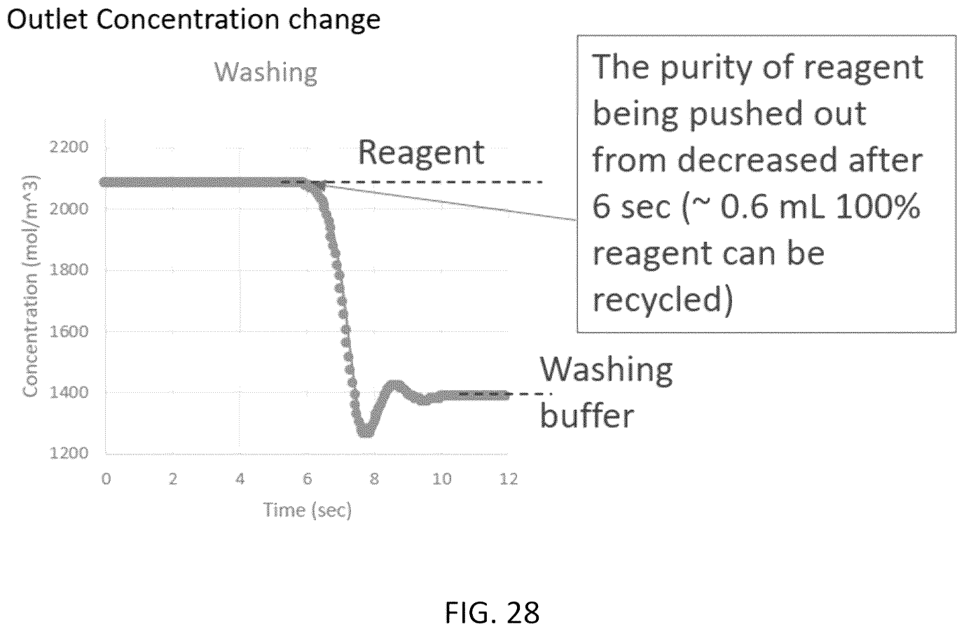

FIG. 28 shows outlet concentration change during a simulated washing cycle. FIG. 28 shows that, for this simulation, after 6 seconds of washing (injecting 0.6 mL of wash surrogate) the purity of reagent surrogate being pushed out of the flow cell begins to decrease. In other words, after 6 seconds of washing, a mixture of both reagent surrogate and wash surrogate will be output from the flow cell. As such, in this simulation, 100% pure reagent can be collected for recycling during the first six seconds of washing.

Using simulations such as this or other techniques, systems like the one illustrated in FIGS. 17-25 may be calibrated such that only pure reagent(s) is/are directed into recycling pathway(s) and wash fluid and wash fluid/reagent mixtures are directed into a waste pathway. In this manner, a majority of the reagent volume used can be recaptured for reuse.

While the principles of the disclosure have been described above in connection with specific examples of flow cells, systems, and methods, it is to be understood that this description is made only by way of example and not as limitation on the scope of the present inventions. Examples were chosen and described in order to explain the principles of the invention and practical applications to enable others skilled in the art to utilize the invention in various implementations and with various modifications, as are suited to a particular use contemplated. It will be appreciated that the description is intended to cover modifications and equivalents.

1. A flow cell system, the system comprising:
(a) a flow cell, the flow cell comprising:
(i) a substrate, the substrate configured to support an analyte array;
(ii) a cover spaced from the substrate, the substrate and the cover defining a fluid gap between the substrate and cover;
(iii) an inlet in fluid communication with the fluid gap; and
(iv) an outer perimeter of the flow cell;
(b) wherein the flow cell system is configured such that fluid enters the flow cell at the inlet, flows through the fluid gap, and exits the flow cell at the outer perimeter between the substrate and the cover.

2. The flow cell system of claim 1, wherein the flow cell system is configured such that fluid exits the flow cell at the outer perimeter of the flow cell between the substrate and the cover such that fluid drops down from the outer perimeter as droplets.

3. The flow cell system of claim 2, wherein the outer perimeter of the flow cell is at least partially open.

4. The flow cell system of claim 3, wherein the outer perimeter is at least 2.5% open.

5. The flow cell system of claim 3, wherein the outer perimeter is at least 5% open.

6. The flow cell system of claim 3, wherein the outer perimeter of the flow cell is open.

7. The flow cell system of claim 3, wherein the outer perimeter of the flow cell is frameless.

8. The flow cell system of claim 7, wherein the flow cell further comprises a spacer spacing the substrate and the cover apart to define the fluid gap.

9. The flow cell system of claim 8, wherein the spacer comprises a plurality of spacers, at least some of the spacers being generally aligned towards a central area of the flow cell.

10. The flow cell system of claim 9, wherein at least some of the spacers extend radially from the central area of the flow cell.

11. The flow cell system of claim 8, wherein the spacer comprises an adhesive arrangement spacing the substrate and the cover apart to define the fluid gap and adhering the substrate to the cover.

12. The flow cell system of claim 3, wherein the inlet is located in the central area of the flow cell.

13. The flow cell system of claim 3, further comprising a common collector, the common collector positioned below the outer perimeter of the flow cell and configured to collect droplets that drop down from the outer perimeter of the flow cell.

14. The flow cell system of claim 13, wherein the common collector comprises a plurality of common collection cups configured to collect droplets that drop down from the outer perimeter of the flow cell.

15. The flow cell system of claim 14, wherein the common collection cups are vertically spaced from the flow cell by a drop gap.

16. The flow cell system of claim 14, wherein the common collection cups each comprise a sloped side wall, wherein a portion of the sloped side wall below the outer perimeter of the flow cell is vertically spaced from the flow cell by a drop gap.

17. The flow cell system of claim 14, wherein at least some of the common collection cups each comprise a sloped wall configured to direct the collected droplets to a fluid conduit.

18. The flow cell system of claim 17, wherein the collection channel comprises a plurality of fluid conduits.

19. The flow cell system of claim 17, wherein the sloped wall comprises a slope that is greater than 30 degrees.

20. A flow cell system, the system comprising:
(a) a flow cell, the flow cell comprising:
(i) a substrate, the substrate configured to support an analyte array;
(ii) a cover;
(iii) an adhesive arrangement spacing the cover from the substrate to define a fluid gap between the substrate and cover;
(iv) an inlet in fluid communication with the fluid gap; and
(v) an outer perimeter of the flow cell;
(b) wherein the flow cell system is configured such that fluid enters the flow cell at the inlet, flows through the fluid gap, and exits the flow cell at the outer perimeter between the substrate and the cover; wherein adhesive gaps in the adhesive arrangement at the outer perimeter of the flow cell allow fluid to exit from the fluid gap.

21. The flow cell system of claim 20, wherein at least five separate adhesive gaps in the adhesive arrangement at the outer perimeter of the flow cell allow fluid to exit from the fluid gap.

22. The flow cell system of claim 20, wherein at least ten separate adhesive gaps in the adhesive arrangement at the outer perimeter of the flow cell allow fluid to exit from the fluid gap.

23. A flow cell system, the system comprising:
(a) a flow cell, the flow cell comprising:
(i) a substrate, the substrate having a surface configured to support an analyte array, the surface configured to support the analyte array having a surface area of at least 150 cm²;
(ii) a cover spaced from the substrate, the substrate and the cover defining a fluid gap between the substrate and cover, the fluid gap having a fluid gap volume that is less than 1.5 mL;
(iii) an inlet in fluid communication with the fluid gap; and
(iv) an outer perimeter of the flow cell;
(b) wherein the flow cell system is configured such that fluid enters the flow cell at the inlet, flows through the fluid gap, and exits the flow cell at the outer perimeter between the substrate and the cover.

24. The flow cell system of claim 23, wherein the surface configured to support the analyte array has a surface area of at least 225 cm² and the fluid gap has a fluid gap volume that is less than 1 mL.

25. The flow cell system of claim 23, wherein the surface configured to support the analyte array has a surface area of at least 300 cm² and the fluid gap has a fluid gap volume that is less than 0.75 mL.

26.-63. (canceled)
